(19) United States
(12) Patent Application Publication
(10) Pub. No.: US 2007/0239148 A1
(43) Pub. Date: Oct. 11, 2007

(54) Laser Probe Assembly with Laser Light Source Connector and Electronic Identification Connector

(75) Inventor: Gregg D. Scheller, Glencoe, MO (US)

Correspondence Address: THOMPSON COBURN, LLP, ONE US BANK PLAZA, SUITE 3500, ST LOUIS, MO, 63101, US

(73) Assignee: SYNERGETICS, INC., St. Charles, MO (US)

(21) Appl. No.: 11/734,109

(22) Filed: Apr. 11, 2007

Related U.S. Application Data
(60) Provisional application No. 60/791,171, filed on Apr. 11, 2006.
(60) Provisional application No. 60/858,053, filed on Nov. 10, 2006.

(51) Int. Cl. A61B 18/18

(52) U.S. Cl. 606/15

(57) ABSTRACT

An optic fiber surgical instrument is removably connectable to a surgical light source. The instrument is provided with an optic fiber connector and a separate auxiliary or electric identification connector. The optic fiber connector is removably connectable to a laser light output of a laser light source to convey the laser light through the optic fiber of the instrument, whereby manipulation of the instrument by a user can direct the laser light to a surgical site. The auxiliary connector is connectable to a ground connection of the surgical light source to establish an electric circuit through the instrument and the surgical light source, whereby an electrical identification device on the instrument identifies the instrument for the surgical light source.

This patent application claims the benefit of provisional patent application No. 60/791,171, filed on Apr. 11, 2006, and provisional patent application Ser. No. 60/858,053, filed on Nov. 10, 2006.

### BACKGROUND OF THE INVENTION

1. Field of the Invention

The present invention pertains to an optic fiber surgical instrument that is removably connectable to a surgical light source. In particular, the instrument is provided with an optic fiber connector and a separate auxiliary or electric identification connector. The optic fiber connector is removably connectable to a laser light output of a laser light source to convey the laser light through the optic fiber of the instrument, whereby manipulation of the instrument by a user can direct the laser light to a surgical site. The auxiliary connector is connectable to a ground connection of the surgical light source to establish an electric circuit through the instrument and the surgical light source, whereby an electrical identification device on the instrument identifies the instrument for the surgical light source.

2. Description of the Related Art

In ophthalmic surgery, various different types of instruments are available for use by the surgeon to deliver light to a surgical site in the interior of the eye. These instruments deliver light for illumination of the surgical site, and also deliver laser light for performing a surgical procedure at the surgical site. A basic instrument of this type is comprised of a handle with a projecting tubular tip and a length of optic fiber that extends through the handle and the tip to a distal end of the optic fiber positioned at the tip distal end. The opposite, proximal end of the optic fiber is provided with a connector for connecting the optic fiber to a light source, either an illumination light source or a laser light source. By connecting the optic fiber connector to the light output of the surgical light source, the light from the surgical light source is conveyed through the fiber to the fiber distal end where the light is emitted from the fiber and directed to the surgical site.

Examples of optic fiber surgical instruments similar to that described above are disclosed in the U.S. Pat. No. 5,085,492 of Kelsoe et al. and the U.S. Pat. Nos. 6,357,932 and 6,634,799 of Auld, the U.S. Pat. No. 6,572,608 of Lee et al., and the U.S. Pat. No. 6,984,230 of Scheller et al. Each of these patents is incorporated herein by reference.

Prior art optic fiber surgical instruments of the type disclosed in the above-referenced U.S. patents include surgical laser light sources. These light sources have female connectors that are designed to attach to the male connectors of a set of optic fiber surgical instruments. The prior art includes a laser light source that is specifically designed to operate only if the light source recognizes an electronic identifier on the surgical instrument connected to the light source. The light source will not operate if a male connector of a microsurgical instrument is connected to the female connector of the light source where the surgical instrument does not have the electronic identifier. This is accomplished by encoding the male connectors of the surgical instruments with an electronic device that is recognized by the light source when the male connector is attached to the female connector of the light source. An example of such a male connector and light source is disclosed in the prior referenced U.S. Pat. No. 5,085,492 of Kelsoe et al.

The above-described prior art laser light source that can only be used with a set of microsurgical instruments that is recognized by the light source has been found to be inconvenient when it is desirable to use other types of surgical instruments with the particular laser light source. This inconvenience could be overcome by an optic fiber surgical instrument that can be attached to the female connector of a laser light source of the type that operates only with associated electronically encoded surgical instruments, where the optic fiber surgical instrument also has an auxiliary connector that can be connected to the laser light source to create a circuit between the optic fiber instrument connector and the laser light source that includes an electronic encoded device that is recognized by the laser light source.

### SUMMARY OF THE INVENTION

The present invention overcomes the inconveniences associated with the prior art laser light source described above by providing an optic fiber surgical instrument that includes an optic fiber connector that can be connected to the female connector of the laser light source, and also includes a separate auxiliary connector that can be grounded to the housing of the laser light source to create an electronic circuit between the female connector of the laser light source and the housing of the laser light source that includes an electronic device recognized by the controls of the laser light source. In addition, the auxiliary connector is provided with circuitry that enables the optic fiber surgical instrument to be used with the laser light source that only recognizes encoded surgical instruments where the laser light source has been wired incorrectly during manufacturing.

The optic fiber surgical instrument of the invention, in the preferred embodiments, has a handpiece that can be manually manipulated by a user of the instrument, and a rigid tubular tip projecting from the handpiece. A length of optic fiber having opposite proximal and distal ends extends through the handpiece and the tip with the optic fiber distal end being positioned adjacent the tip distal end. The optic fiber can be secured stationary relative to the handpiece and tip, or can be movable relative to the handpiece and tip. A majority of the optic fiber length extends from the handpiece to the optic fiber proximal end.

An optic fiber connector is provided on the optic fiber proximal end. The optic fiber connector includes a center ferrule and an internally threaded nut that is designed to be threaded to a threaded bushing at the laser light output of the surgical laser light source. The proximal end of the optic fiber is secured inside the ferrule. The ferrule is inserted into the center of the laser light source bushing, and the threaded nut on the optic fiber connector is screw threaded onto the bushing to positively position the proximal end of the optic fiber relative to a beam of laser light delivered by the surgical laser light source.

An electrical conductor extends from the optic fiber connector. The electrical conductor has a length with opposite proximal and distal ends, and the distal end of the electrical conductor is operatively electrically connected to the ferrule of the optic fiber connector. By being operatively electrically connected to the ferrule, what is meant is that the wire distal end is connected as part of an electric circuit with the ferrule of the optic fiber connector. The wire can either be directly, physically connected to the ferrule, or the wire can be in electrical communication with the ferrule through the intermediary of other electrical conductors between the wire and the ferrule. The opposite proximal end of the electrical conductor is operatively electrically connected to an auxiliary connector or an electrical identification connector.

The auxiliary connector is designed to provide an electrical connection to a ground of the surgical light source, typically the housing of the surgical light source. This ground connection can be provided by a simple mechanical connection between the wire proximal end and the light source housing. In the preferred embodiment, the auxiliary connector is a conventional RCA jack that is connected to an existing RCA plug on the light source. For example, the RCA jack can be connected to an existing head lamp connector typically provided on the prior art laser light source. The RCA jack is pressed onto the head lamp RCA plug connector of the laser light source to establish an electric circuit from the bushing of the laser light source, through the optical fiber connector, through the electrical conductor, through the auxiliary connector, to the head lamp connector on the laser light source housing.

An electrical identification device is operatively electrically connected along the conductor that connects the optical fiber connector with the auxiliary connector. In one embodiment, the electrical identification device is a resistor that is connected in series between two portions of a length of wire of the electrical conductor. The resistor, connected in the electrical circuit from the light source bushing to the light source ground, is recognized by the light source and enables the optic fiber surgical instrument to be used with the light source when the optical fiber connector is connected to the bushing and the auxiliary connector is connected to the light source housing ground connection, i.e., the head lamp connector. In other embodiments, the electrical identification device could be some other type of identification device. For example, the electrical identification device could be some type of capacitor, some type of inductor, or some type of diode. In addition, the electrical identification device could be a memory chip. It is also possible for the electrical identification device to be a serial data device that communicates by rapidly changing the current flow through the electrical identification device to represent 1s and 0s. Such a device could be powered by the voltage present on the ferrule receptacle of the laser light source and would only require the connection of the instrument ferrule to the receptacle and the ground connection provided by the RCA connector. Still further, a zener diode could be used as the electrical identification device. Such a diode would break down at a pre-determined reverse voltage and appear as a specific sized resistor to the identification control system circuit in the laser light source.

In an alternate embodiment of the optic fiber surgical instrument, the electrical identification device is connected between the proximal end of the electrical conductor and the sleeve of the RCA auxiliary connector.

In a further alternate embodiment of the invention, the electrical identification device is operatively electrically connected to the electrical conductor proximal end, and is operatively electrically connected to the sleeve of the RCA auxiliary connector through a first diode, and is operatively electrically connected to the center post of the RCA auxiliary connector through a second diode. With this wiring arrangement, regardless of how the auxiliary connector plug or head lamp connector plug on the surgical light source is wired and grounded, a grounding connection to the housing of the surgical light source is provided by connecting the RCA auxiliary connector jack to the head lamp connector plug of the light source.

### BRIEF DESCRIPTION OF THE DRAWINGS

Further features of the invention are set forth in the following detailed description of the preferred embodiments of the invention and in the drawing figures.

FIG. 1 is a partially sectioned view of a first embodiment of the optic fiber surgical instrument of the invention.

FIG. 2 is a partially sectioned view of a second embodiment of the optic fiber surgical instrument of the invention.

FIG. 3 is a partially sectioned view of a third embodiment of the optic fiber surgical instrument of the invention.

FIG. 4 is a schematic representation of the electronic encoded circuit of FIG. 3.

### DETAILED DESCRIPTION OF THE PREFERRED EMBODIMENTS

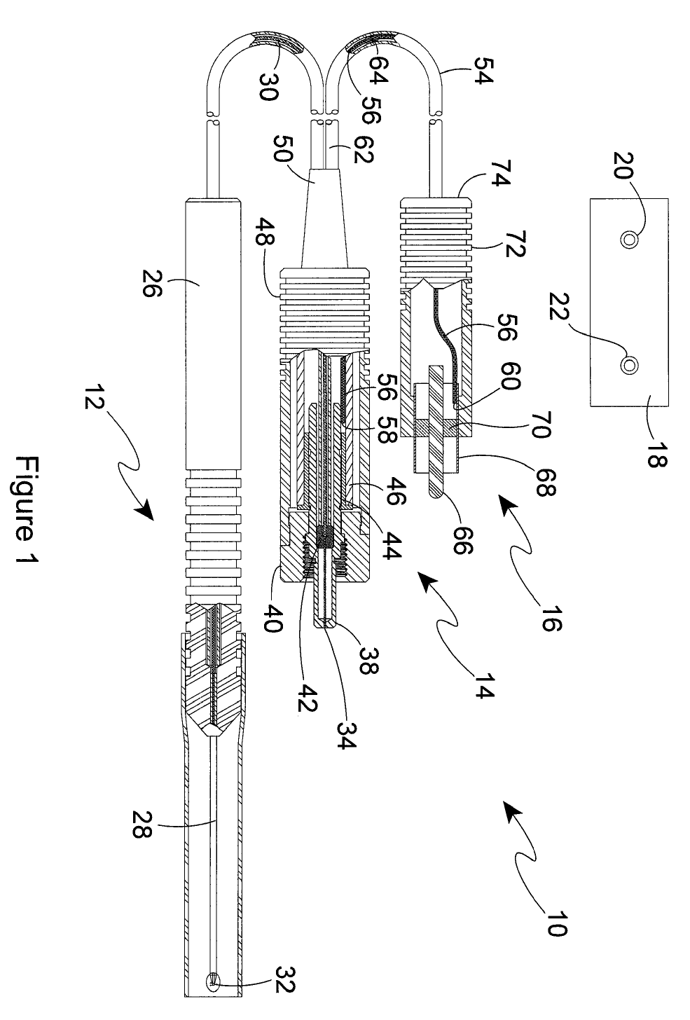

One embodiment of the optic fiber surgical instrument 10 of the present invention is shown in FIG. 1. This instrument can generally be described as a laser probe assembly 12 with a light source connector 14 and an electronic identification connector 16. Although the instrument 10 shown in FIG. 1 includes a laser probe 12, it should be understood that the concept of the invention can be used in any type of surgical instrument that employs a length of optic fiber that communicates illumination light or laser light to a surgical site from a surgical light source. In FIG. 1 the surgical light source 18 is represented schematically. The light source 18 is similar to that disclosed in the previously referenced U.S. Pat. No. 5,085,492 of Kelsoe et al. A prior art light source of this type typically includes an externally threaded bushing that functions as the light source connector 20, and a RCA plug 22 that functions as an auxiliary connection, typically for a power connection for a separate headlamp used by the surgeon during surgical procedures. The RCA plug 22 connects to a typical RCA jack, with the jack having a hot connection and a ground connection. Thus, the RCA plug 22 functions as a ground connection to the housing of the surgical light source 18.

In the embodiment of the instrument shown in FIG. 1, the instrument has a handpiece 26 that can be manually manipulated by a user of the instrument. A ridged tubular tip 28 projects from the handpiece. A length of optic fiber 30 having opposite distal 32 and proximal 34 ends extends through the handpiece 26 and through the tip 28 to the optic fiber distal end 32 positioned adjacent the tip distal end. Ophthalmic instruments of this type are known in the art. As in prior art instruments, the optic fiber 30 can be secured stationary relative to the handpiece 26 and the tip 28. Alternatively, a mechanism can be provided on the handpiece 26 for selectively moving the optic fiber 30 through the handpiece 26 and the tip 28. As is also known in the prior art, the optic fiber 30 could be held stationary relative to the handpiece 26, and a mechanism could be provided on the handpiece for selectively moving the tip 28 relative to the handpiece 26 and the optic fiber distal end 32. Although the optic fiber length 30 is shown fragmented in FIG. 1, a majority of the optic fiber length extends from the handpiece 26 to the optic fiber proximal end 34. This elongated length of the optic fiber 30 enables the user of the instrument to freely manipulate the handpiece 26 independently of the optic fiber proximal end 34.

The light source connector or optic fiber connector 14 is provided on the optic fiber adjacent the optic fiber proximal end 34. The optic fiber connector 14 shown in FIG. 1 is only one example of an optic fiber connector that may be used with the instrument of the invention. Because the optic fiber connector 14 can have different constructions other than that shown, the construction shown is described only generally herein. Basically, the optic fiber connector 14 includes a center ferrule 38 and an internally threaded nut 40 mounted on the ferrule. The ferrule 38 is constructed of electrically conductive materials. The internally threaded nut 40 is constructed of plastic and has internal screw threads that are complementary to the external screw threads of the laser light connector bushing 20 of the laser light source with which the instrument 10 is to be used. The ferrule 38 has a center hole at an end of the ferrule that receives the proximal end 34 of the optic fiber 30. A bushing 42 is provided in the interior of the ferrule 38 that securely holds the optic fiber 30 in the ferrule and centers the optic fiber relative to the interior of the ferrule. A retainer 44 is mounted on the ferrule 38 adjacent the nut 40. The retainer 44 limits the axial movement of the nut 40 on the ferrule 38, and allows the nut 40 to rotate on the ferrule 38. A resilient, strain relief boot 46 is secured around the retainer 44. A cylindrical sleeve 48 is secured to the nut 40 and functions as an extension of the nut. The opposite end of the boot 46 from the nut 40 has a tapered configuration 50. The length of optic fiber 30 enters the optic fiber connector 14 through the tapered end 50 of the boot.

An electrical conductor 54 is connected to the optic fiber connector 14. The electrical conductor 54 comprises a single length of electrically conductive wire 56 having opposite distal 58 and proximal 60 ends. In alternative embodiments, multiple electrical conductive wires could be employed instead of the single wire 56. The wire distal end 58 is operatively, electrically connected to the optic fiber ferrule 38. The electrical connection is provided by the wire distal end 58 being secured both physically and electrically to the optic fiber connector ferrule 38. The wire exits the optic fiber connector 14 through the tapered end 50 of the boot 46. The portion of the wire 56 outside of the optic fiber connector 14 is protected by an exterior insulating layer 62.

In the embodiment of the instrument shown on FIG. 1, the electrical conductor 54 includes an electrical identification device 64 that is secured in series between two portions of the length of the conductor wire 56. In this embodiment the electrical identification device 64 is a resistor. However, it should be understood that other types of electrical identification devices could be used. In other embodiments, the electrical identification device could be some other type of identification device. For example, the electrical identification device could be some type of capacitor, some type of inductor, or some type of diode. In addition, the electrical identification device could be a memory chip. It is also possible for the electrical identification device to be a serial data device that communicates by rapidly changing the current flow through the electrical identification device to represent 1s and 0s. Such a device could be powered by the voltage present on the ferrule receptacle of the laser light source and would only require the connection of the instrument ferrule to the receptacle and the ground connection provided by the RCA connector. Still further, a zener diode could be used as the electrical identification device. Such a diode would break down at a pre-determined reverse voltage and appear as a specific sized resistor to the identification control system circuit in the laser light source. The resistor 64 is chosen as the electrical identification device that is recognized by the particular surgical light source 18 with which the instrument is to be used.

The electrical conductor proximal end 60 is secured to the auxiliary connector 16. The length of the electrical conductor 54 allows the auxiliary connector 16 to be freely manually moved relative to the optic fiber connector 14. In the embodiment shown on FIG. 1, the auxiliary connector 16 is a standard RCA type connector. As is typical of this type of connector, the auxiliary connector 16 has an electrically conductive center post 66, and electrically conductive cylindrical sleeve 68 surrounding the center post. A ring 70 of electrically insulating material surrounds the center post 66 and secures the sleeve 68 around the center post. The wire 56 at the electrical conductor proximal end 60 is operatively electrically connected to the sleeve 68 of the auxiliary connector 16. An electrically insulating strain relief boot 72 is secured around the exterior of the sleeve 68 and extends around the electrical conductor proximal end 60. The opposite end 74 of the strain relief boot 72 from the connector sleeve 68 is provided with a small hole through which the electrical conductor 54 passes.

In use of the instrument 10 of FIG. 1 with the laser light source 18, the ferrule 38 of the optic fiber connector 14 is inserted into the center of the laser light source bushing 20 and the threaded nut 40 is screw threaded on the exterior screw threading of the bushing. This positively positions the optic fiber proximal end 34 relative to a beam of laser light delivered by the surgical light source 18.

With the optic fiber connector 14 secured to the surgical light source 18, the length of the electrical conductor 54 enables the free movement of the auxiliary connector 16 to position the auxiliary connector 16 on the auxiliary connection 22 of the laser light source. The RCA jack of the auxiliary connector 16 is press fit on the RCA plug 22 of the laser light source 18. This establishes a grounding connection of the wire 56 at the electrical conductor proximal end 60 to the laser light source 18. With the optic fiber connector 14 and the auxiliary connector 16 connected to the laser light source 18 in this matter, an electrical circuit is established from the laser light source bushing 20, through the optic fiber connector ferrule 38, the wire 56 of the electrical conductor which includes the electrical identification device 64, the sleeve 68 of the auxiliary connector 16, and the RCA plug 22 of the surgical light source 18 which is typically connected to an electrical ground of the light source. The electric circuit is completed through a control system of the laser light source 18 which provides for electrical communication of the laser light source bushing 20 to the auxiliary connection 22. This electric circuit enables the electrical identification device 64 to be recognized by the control system of the laser light source 18, and enables the use of the instrument 10 with the laser light source.

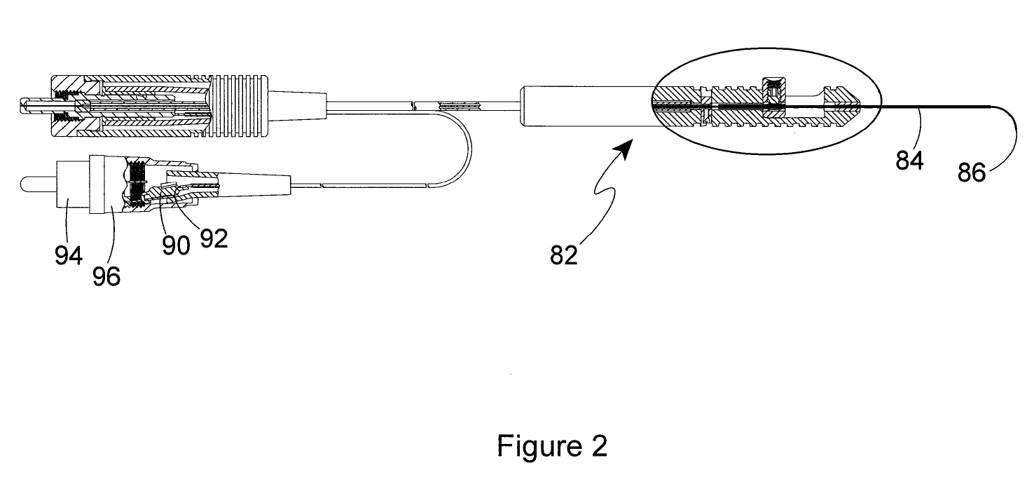

FIG. 2 shows a second embodiment of the instrument of the invention. In the embodiment of FIG. 2, the laser probe 82 is slightly different than the laser probe 12 of FIG. 1. The laser probe 82 of FIG. 2 is of a type described earlier, where the instrument tip 84 can be selectively moved relative to the optic fiber distal end 86. This enables the optic fiber distal end 86 to be moved through a gradual bend, and enables the directing of the laser light emitted from the optic fiber distal end. Instruments of this type are known in the art.

The primary difference between the embodiment of FIG. 2 and the embodiment of FIG. 1 is the repositioning of the electrical identification device 90 to the proximal end 92 of the electrical conductor wire. The electrical identification device 90 is connected between the wire proximal end 92 and the RCA connector sleeve 94 of the auxiliary connector 96. As in the first described embodiment, the electrical identification device 90 is operatively electrically connected between the electrical conductor wire proximal end 92 and the auxiliary connector sleeve 94. Thus, the electrical identification device 90 operates in the same manner as the embodiment of FIG. 1.

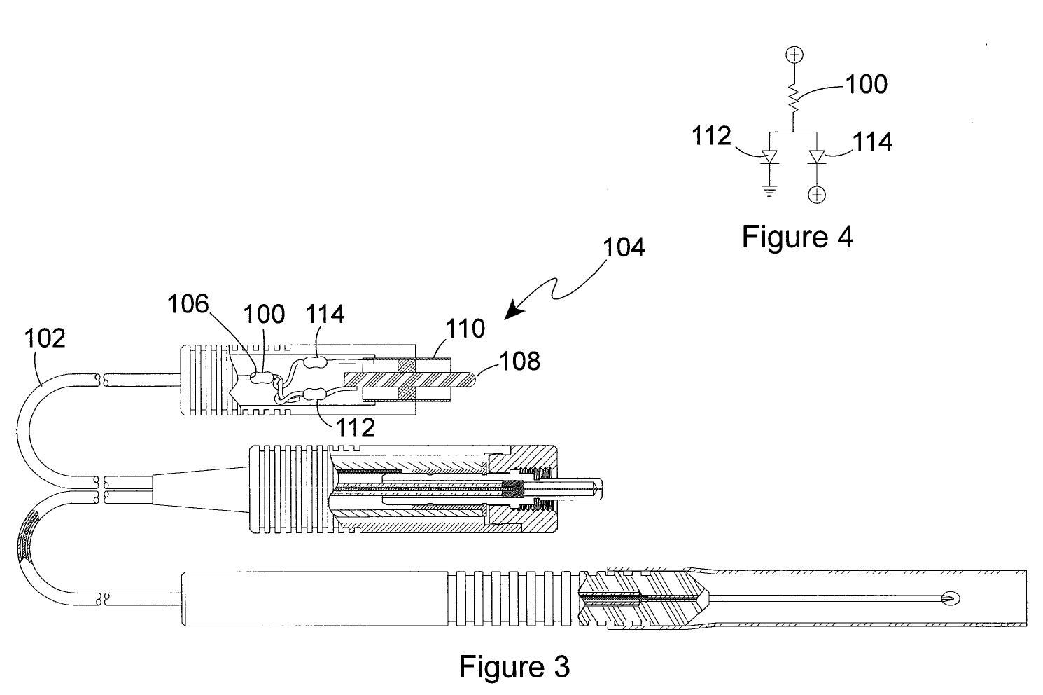

FIG. 3 shows the third embodiment of the instrument that is similar to that of FIG. 1. In the embodiment of FIG. 3, the electrical identification device 100 is moved from the length of the electrical conductor 102 into the interior of the auxiliary connector 104. As in the previously described embodiment, the electrical identification device 100 is operatively electrically connected to the wire proximal end 106 of the electrical conductor 102. However, the embodiment of FIG. 3 differs from that of FIGS. 1 and 2 in that the electrical identification device 100 is also operatively electrically connected to the center post 108 of the RCA auxiliary connector 104, and to the sleeve 110 of the RCA auxiliary connector. The electrical identification device 100 is operatively electrically connected through a first diode 112 to the auxiliary connector center post 108, and is operatively electrically connected through a second diode 114 to the auxiliary connector sleeve 110. FIG. 4 shows a schematic representation of the portion of the circuit defined by the electrical identification device 100 and the first 112 and second 114 diodes. The embodiment of the instrument shown in FIG. 3, and in particular the auxiliary connector 104 was developed for use with a laser light source such as that of FIG. 1 that has an alternative wiring of the RCA connector 22 of the light source.

In the typical laser light source such as that shown in FIG. 1, the light source is provided with a headlamp power output receptacle in the form of an RCA plug. The outer conductive sleeve of the RCA plug is connected to a ground of the laser light source. The center terminal of the RCA plug is at low voltage potential to operate a headlamp when an RCA jack of the headlamp is inserted into the RCA plug.

It has been discovered that a significant number of laser light sources have been wired in reverse, i.e., with the center terminal of the RCA plug being an electrical ground and with the outer sleeve of the RCA plug at a low voltage potential. As a result, as to those reversed wired electric light sources, the laser instrument of FIGS. 1 and 2 would not operate because the auxiliary connectors 16, 96 would not be provided with the necessary electrical ground. To address this problem, the modified auxiliary connector 104 of FIG. 3 was designed. This connector 104 connects the electrical identification device 100 to both the center post 108 and the sleeve 110 through the first 112 and second 114 diodes, respectively. In this way, the one side of the electrical identification device 100 is connected to an electrical ground upon connecting the auxiliary connector 104 to the headlamp power output RCA plug 22 of the laser light source, no matter which of the center post 108 or sleeve 110 is electrically grounded. The diode 112, 114 in the other branch of the circuit shown in FIG. 4 blocks current from the RCA post 108 or sleeve 110 connected to the RCA plug 22 that is at voltage potential. Thus, regardless of whether the RCA plug 22 has been incorrectly wired, an electric circuit is established from the laser light source bushing, through the optic fiber connector, the electrical conductor 102, the electrical identification device 100, and one of the two diodes 112, 114 to the electrical ground of the RCA plug 22.

Thus, the laser probe assembly with the laser light source connector and the electronic identification connector of the invention provides an alternative optic fiber surgical instrument that can be used with a laser light source that requires an electronic identification of the instrument used with the laser light source.

Although the laser probe assembly of the invention has been described above by reference to specific embodiments, it should be understood that the modifications and variations to the probe assembly could be arrived at without departing from the intended scope of the following claims.

What is claimed is:

1. An optic fiber surgical instrument that is connectable to a surgical light source, the instrument comprising:
a length of optic fiber having opposite proximal and distal ends, the optic fiber proximal end being adapted for receiving light to convey the light through the optic fiber length to the optic fiber distal end where light conveyed through the optic fiber length is emitted from the optic fiber distal end to be directed to a surgical site;
an optic fiber connector connected to the optic fiber adjacent the optic fiber proximal end, the optic fiber connector being connectable to a surgical light source to enable the optic fiber proximal end to receive light from the surgical light source; and,
an auxiliary connector that is operatively electrically connected to the optic fiber connector, the auxiliary connector being separate from the optic fiber connector and being connectable to the surgical light source separate from the optic fiber connector when the optic fiber connector is connected to the surgical light source to establish an electric circuit from the surgical light source, through the optic fiber connector, through the auxiliary connector and to the surgical light source.

2. The instrument of claim 1, further comprising:
a hand piece connected to the optic fiber adjacent the optic fiber distal end, the hand piece being manually manipulatable to manually direct light emitted from the optic fiber distal end to a surgical site.

3. The instrument of claim 1, further comprising:
an electrical conductor having a length with opposite proximal and distal ends, the electrical conductor distal end being operatively electrically connected to the optic fiber connector and the electrical conductor proximal end being operatively electrically connected to the auxiliary connector where the electrical conductor length enables the optic fiber connector and the auxiliary connector to be spaced apart.

4. The instrument of claim 3, further comprising:
the electrical conductor length enabling relative movement between the optic fiber connector and the auxiliary connector.

5. The instrument of claim 3, further comprising:
the optic fiber connector being operatively electrically and removably connectable to a light source connector of the surgical light source; and,
the auxiliary connector being operatively electrically and removably connectable to a ground connection of the surgical light source.

6. The instrument of claim 5, further comprising:
the optic fiber connector, the electrical conductor, and the auxiliary connector defining an electric circuit from the light source connector of the surgical light source, through the optic fiber connector, through the electrical conductor, through the auxiliary connector and to the ground connection of the surgical light source when the optic fiber connector is connected to the light source connector and the auxiliary connector is connected to the ground connector.

7. The instrument of claim 5, further comprising:
the length of the electrical conductor enabling the auxiliary connector to be manually moved relative to the optic fiber connector and enabling the optic fiber connector to be manually moved relative to the auxiliary connector.

8. The instrument of claim 7, further comprising:
an electrical identification device operatively electrically connected to the electrical conductor.

9. The instrument of claim 8, further comprising:
the electrical identification device being a resistor.

10. The instrument of claim 8, further comprising:
the electrical identification device being at least one diode.

11. The instrument of claim 3, further comprising:
the auxiliary connector having a center electrically conductive pin, a cylindrical electrically conductive sleeve surrounding the center pin, and an electrical insulator separating the center pin and the sleeve; and,
the electrical conductor proximal end being operatively electrically connected to at least one of the center pin or the sleeve.

12. The instrument of claim 11, further comprising:
the electrical conductor proximal end being operatively electrically connected to both the center pin and the sleeve.

13. The instrument of claim 12, further comprising:
a first diode that is operatively electrically connected between the electrical conductor proximal end and the center pin; and,
a second diode that is operatively electrically connected between the electrical conductor proximal end and the sleeve.

14. The instrument of claim 13, further comprising:
an electrical identification device that is operatively electrically connected between the electrical conductor proximal end and the first diode and is operatively electrically connected between the electrical conductor proximal end and the second diode.

15. An optic fiber surgical instrument that is connectable to a surgical light source, the instrument comprising:
at least one optic fiber having an optic fiber length with opposite optic fiber proximal and optic fiber distal ends, the optic fiber proximal end being adapted for receiving light to convey the light through the optic fiber length to the optic fiber distal end where conveyed light is emitted and directed to a surgical site;
a hand piece on the at least one optic fiber adjacent the optic fiber distal end, the hand piece being manually manipulatable to direct the optic fiber distal end;
an optic fiber connector on the at least one optic fiber adjacent the optic fiber proximal end, the optic fiber connector being removably connectable to a surgical light source to position the optic fiber proximal end to receive light from the surgical light source;
an electrical conductor having a length with opposite proximal and distal ends, the electrical conductor distal end being operatively electrically connected to the optic fiber connector; and,
an auxiliary connector that is removably connectable to the surgical light source, the auxiliary connector having a center electrically conductive pin, a cylindrical electrically conductive sleeve, and an electric insulator electrically insulating the pin and sleeve, the electrical conductor proximal end being operatively electrically connected to at least one of the pin and the sleeve.

16. The instrument of claim 15, further comprising:
the electrical conductor length enabling relative movement between the optic fiber connector and the auxiliary connector.

17. The instrument of claim 15, further comprising:
an electrical identification device operatively electrically connected to the electrical conductor.

18. The instrument of claim 17, further comprising:
the electrical identification device being a resistor.

19. The instrument of claim 17, further comprising:
the electrical identification device being at least one diode.

20. The instrument of claim 15, further comprising:
the electrical conductor proximal end being operatively electrically connected to both the center pin and the sleeve.

21. The instrument of claim 20, further comprising:
a first diode that is operatively electrically connected between the electrical conductor proximal end and the center pin; and,
a second diode that is operatively electrically connected between the electrical conductor proximal end and the sleeve.

22. The instrument of claim 21, further comprising:
an electrical identification device that is operatively electrically connected between the electrical conductor proximal end and the first diode and is operatively electrically connected between the electrical conductor proximal end and the second diode.

23. An optic fiber surgical instrument that is connectable to a separate surgical light source, the instrument comprising:
a manually manipulatable hand piece, a narrow tubular tip projecting from the hand piece, and an optic fiber extending through the hand piece and the tip, the optic fiber having a length with opposite proximal and distal ends, and a portion of the optic fiber length extending through the hand piece and through the tip to the optic fiber distal end;
an optic fiber connector at the optic fiber proximal end, the length of the optic fiber enabling the hand piece to be manually moved relative to the optic fiber connector, the optic fiber connector being connectable to a light source connector of the surgical light source, the optic fiber connector having a center ferrule that holds the optic fiber proximal end at a predetermined position relative to the surgical light source when the optic fiber connector is connected to the light source connector of the surgical light source;
an electrical conductor having a length with opposite proximal and distal ends, the electrical conductor distal end being operatively electrically connected to the optic fiber connector; and,
an auxiliary connector operatively electrically connected to the electrical conductor proximal end, the length of the electrical conductor enabling the auxiliary connector to be manually moved relative to the optic fiber connector, the auxiliary connector being adapted for connection to the surgical light source.

24. The instrument of claim 23, further comprising:
the optic fiber connector, the electrical conductor, and the auxiliary connector defining an electric circuit from the light source connector of the surgical light source, through the optic fiber connector, through the electrical conductor, through the auxiliary connector and to the surgical light source when the optic fiber connector is connected to the light source connector and the auxiliary connector is connected to the surgical light source.

25. The instrument of claim 23, further comprising:
an electrical identification device operatively electrically connected to the conductor.

26. The instrument of claim 25, further comprising:
the electrical identification device being a resistor.

27. The instrument of claim 25, further comprising:
the electrical identification device being at least one diode.

28. The instrument of claim 23, further comprising:
the auxiliary connector being adapted for connection to the surgical light source to electrically ground the electrical conductor to the surgical light source.

29. The instrument of claim 23, further comprising:
the optic fiber connector being adapted for being removably connected to the light source connector of the surgical light source; and,
the auxiliary connector being adapted for being removably connected to the surgical light source.

30. The instrument of claim 23, further comprising:
the auxiliary connector having a center electrically conductive pin, a cylindrical electrically conductive sleeve surrounding the center pin, and an electric insulator separating the center pin from the sleeve; and,
the electrical conductor proximal end being operatively electrically connected to at least one of the center pin or sleeve.

31. The instrument of claim 30, further comprising:
the electrical conductor proximal end being operatively electrically connected to both the center pin and the sleeve.

32. The instrument of claim 30, further comprising:
a first diode that is operatively electrically connected between the electrical conductor proximal end and the center pin; and,
a second diode that is operatively electrically connected between the electrical conductor proximal end and the sleeve.

33. The instrument of claim 32, further comprising:
an electrical identification device that is operatively electrically connected between the electrical conductor proximal end and the first diode and is operatively electrically connected between the electrical conductor proximal end and the second diode.
